(19) United States
(12) Patent Application Publication
(10) Pub. No.: US 2007/0021832 A1
(43) Pub. Date: Jan. 25, 2007

(54) Foldable intraocular lens with adaptable haptics

(76) Inventor: Lee T. Nordan, Rancho Santa Fe, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/480,050

(22) Filed: Jun. 30, 2006

Related U.S. Application Data
(60) Provisional application No. 60/696,380, filed on Jul. 1, 2005.

(51) Int. Cl. A61F 2/16

(52) U.S. Cl. 623/6.31; 623/6.44; 623/6.36; 623/6.49

(57) ABSTRACT

A foldable low-compression intraocular lens configured for installation into the anterior chamber of a phakic, pseudophakic or aphakic eye, or a combination thereof. The lens is preferably rolled for insertion through a small corneal incision. Preferably, the implant comprises a resiliently flexible haptic body, an optical lens, and haptics that position the lens within the anterior chamber without excessive compressive forces. The disclosed lens uses refractive or diffractive optics, or a combination of both. One embodiment of the lens contains an optical lens that uses a multi-order diffractive (MOD) structure. An advantage of the disclosed lens is that a single sized device will fit any eye, thus the disclosed invention provides a “one-size-fits-all” intraocular lens.

### CROSS-REFERENCE TO RELATED APPLICATIONS

This application claims the benefit of U.S. provisional patent application Ser. No. 60/696,380 filed Jul. 1, 2005, the disclosures of which are herein incorporated by reference in their entirety.

### TECHNICAL FIELD

The disclosed invention relates to a foldable intraocular lens implant incorporating a prosthetic optical lens and a flexible haptic structure which adapt to the internal dimensions of the eye in which the lens is installed. The disclosed lens adapts to each individual eye with reduced circumferential compressive forces by the inner wall of the anterior chamber. Monofocal, bifocal, and multifocal optics of refractive and diffractive types are contemplated for use with the disclosed invention.

### BACKGROUND ART

Intraocular lenses (IOLs) can be used to correct vision abnormalities. In 1949 Sir Harold Ridley made an artificial lens that he implanted in the eye of a cataract patient. The procedure worked, although Ridley's original lens design was rigid, painful and caused or contributed to glaucoma. Since Sir Ridley's pioneering work, the intraocular lens or “IOL” has evolved and is now commonly used to treat cataract patients.

Intraocular lens come in a variety of shapes and sizes. In general, all IOLs share two components, an optical lens, which is used to enhance or restore visual acuity and a haptic structure, which is used to hold the lens in a fixed position within the patient's eye. Haptic structures (“haptics”) come in a variety of designs. For example, the haptics disclosed in U.S. Pat. No. 6,224,628, “Haptics for an Intraocular Lens,” contemplate a stem structure extending from the optical lens, that transitions to a crossbar extending perpendicular to the stem. A bar with a bulbous end then extends from the crossbar in directions generally parallel to the direct of the stem. Another haptic design is described by U.S. Pat. No. 6,475,240, “Anterior Chamber Intraocular Lens and Method for Reducing Pupil Ovalling,” which describes a system of haptics comprising equally spaced stems that extend radially away from the center of the optical lens, where each stem terminates in a footplate that touches the inner wall of the anterior chamber of the eye. U.S. Pat. No. 6,517,577, “Crossed Haptics for Intraocular Lenses,” describes haptics for IOLs having pairs of haptic stems extending from a haptic base at obtuse angles, typically greater than 100° degrees relative to the center longitudinal axis of the lens. U.S. Pat. No. 6,616,693, “Flexible Fixation Members for Angle-Supported Anterior Chamber Intraocular Lenses,” provides for haptics with rectangular haptic footplates placed approximately 90° degrees from the haptic base. U.S. patent application Ser. No. 10/394,906, Publication No. US 2004/0186568 A1, “Foldable Angle-Fixed Intraocular Lens,” describes an IOL very similar to that disclosed in U.S. Pat. No. 6,616,693, except that the footplates in the application are bulbous and are positioned at about 80° degrees. U.S. patent application Ser. No. 09/794,990, Publication No. US 2002/0120331 A1, “Refractive Anterior Chamber Intraocular Implant,” describes a variety of IOL configurations in combination with a haptic footplate extension pad that is intended to help anchor the IOL to the eye of the patient. U.S. patent application Ser. No. 10/394,906, Publication No. US 2003/0199978 A1, “Stable Anterior Chamber Phakic Lens,” describes an IOL with haptic stems extending away from a haptic base at angles of approximately 65° degrees. U.S. patent application Ser. No. 10/918,078, Publication No. US 2005/0021140 A1, “Accommodating Intraocular Lens with Textured Haptics,” describes an IOL° with haptic placed perpendicular to the stem extending from the optic and at about degrees 70° degrees from the haptic base. Optical lenses and haptic structures may be formed from a common piece of material, or assembled from component parts.

Installation of an IOL may occur in the anterior chamber whether or not a natural, crystalline lens is present. If the natural crystalline lens is absent, an IOL may alternately be implanted in the lens capsule. For either case, it is desirable for an IOL to be small enough to pass through a minimal corneal incision for implantation, in order to reduce the likelihood of subsequent corneal distortion and other surgical side effects or complications.

A limiting factor governing the size of the corneal incision is the diameter of the optical lens required to accommodate a range of pupil diameters for various ambient light levels. Glare and other distortions are likely to occur if a prosthetic optical lens is not large enough to cover a fully dilated pupil for proper nighttime vision. One approach to reducing glare while at the same time reducing the size of the incision in the cornea is to construct the IOL from several pieces which are joined together after the individual pieces are inserted through the corneal incision as disclosed in U.S. Pat. No. 5,769,889, to Charles D. Kelman. The complexity of this type of IOL makes them difficult to install and still results in compromises between reduced incision size and peripheral glare coupled with impaired night vision.

Nordan and Morris improved on the work by Kelman by developing a thin foldable intraocular implant specifically configured for installation into the lens capsule of a phakic (having no natural crystalline lens) or the anterior chamber of a pseudophakic (having a natural crystalline lens) eye having broad haptic flaps with extended contact regions providing reduced peak pressure against the wall of the eye, but reducing the flow of aqueous humor in the anterior capsule resulting in possible complications. (See, U.S. Patent Publication Nos. 20030220687 and 20030097176, and WO 02/41806, which are hereby incorporated by reference in their entirety.) Installation of the disclosed intraocular lenses (IOLs) involves rolling and inserting the lens through a small corneal incision.

Multi-order diffractive (MOD) lenses are useful for bringing a plurality of spectral components of different wavelengths to a common focus. (See, U.S. Pat. No. 5,589,982, to Faklis and Morris, which is hereby incorporated by reference in its entirety.) A MOD lens typically comprises multiple annular zones having step heights defining zone boundaries, which can diffract light of each of the wavelengths in a different diffractive order to a common focus.

### SUMMARY OF THE INVENTION

The invention described herein provides a low-compression, foldable intraocular lens to provide vision correction that allows for the flow of nutrient bearing fluids in the anterior chamber. Monofocal, bifocal, and polyfocal lenses are contemplated by the disclosed invention. The described invention can be used with refractive and diffractive lenses. In one embodiment, a multi-order diffractive lens is used for the intraocular lenses disclosed (IOL).

The disclosed lens can be used with refractive or diffractive optics. One embodiment of the disclosed invention is a low-compression IOL using a monofocal multi-order diffractive (MOD) corrective lens. Such a low-compression IOL comprises a monofocal lens body defining an aperture and wherein the lens comprises a multi-order diffractive structure having a plurality of zones which define zone boundaries at which light incident on the structure experiences an optical phase shift, and diffracts light of each of the wavelengths in a different diffractive order, m, such that m≧1, to said focus, thereby providing a plural order diffractive singlet.

### BRIEF DESCRIPTION OF THE DRAWINGS

FIG. 1a shows a top perspective view of a low-compression intraocular lens (IOL) according to a embodiment of the invention.

FIG. 1b shows an end perspective view of the IOL of FIG. 1a.

FIG. 2 shows a cross-sectional view of the anatomy of a human eye.

FIG. 3 illustrates a cross-sectional view of an IOL implanted in the anterior chamber of a human eye, according to an embodiment of the invention.

FIG. 4a illustrates a top view of an IOL according to an embodiment of the invention.

FIG. 4b illustrates a side view of the IOL shown in FIG. 4a.

FIG. 5a illustrates, in a schematic top view, the position and shape of an IOL according to an embodiment of invention, prior to implantation into the anterior chamber of a human eye.

FIG. 5b illustrates, in a schematic top view, the position and shape of an IOL according to the embodiment of FIG. 5a, subsequent to implantation into the anterior chamber of a human eye.

FIG. 6a illustrates, in a schematic side view, the IOL of FIG. 5a.

FIG. 6b illustrates, in a schematic side view, the IOL of FIG. 5b.

FIG. 7a illustrates in a schematic top view, the position and shape of an IOL according to an alternative embodiment of the invention, prior to implantation into the anterior chamber of a human eye.

FIG. 7b illustrates a schematic side view of the embodiment of FIG. 7a.

FIG. 8a illustrates a schematic side view of the embodiment of FIG. 7a prior to implantation into the anterior chamber of a human eye.

FIG. 8b illustrates a schematic side view of the embodiment of FIG. 7b subsequent to implantation into the anterior chamber of a human eye.

FIG. 9 illustrates a force versus compression characteristic for IOL embodiments.

FIGS. 10a through 10d illustrate embodiments of IOLs with different optical lens positions.

### DETAILED DESCRIPTION OF THE INVENTION

The disclosed invention contemplates a foldable, low-compression intraocular lens (IOL) for implantation into an eye for the correction of refractive errors. Traditional IOLs are compressive in nature in that they rely upon the compression of haptic structures by an inner wall of the eye to frictionally maintain the IOL position within the eye. In a traditional IOL, such compressive forces typically distort the structure of the implanted IOL, causing the optical lens to vault away from the iris and toward the corneal epithelium.

Relying upon a compressive mechanism for placement of the lens within the anterior chamber of the eye is problematic for a number of reasons. Because not all human eyes have the same anterior chamber diameter a clinician installing an intraocular lens in the anterior chamber is forced to engage in a method of trial-and-error lens fitting to select an intraocular lens that is sized appropriately for a particular patient. If the lens fit is too loose, the lens may not be properly fixed in position. If the lens fit is too tight, excessive vaulting of the implant structure can offset the lens position along the optical axis, thereby impairing optical performance. Also, if the lens fits too tightly, the shape of the cornea can be distorted, also potentially causing optical impairment, in addition to potential problems arising from too much contact force against the inner wall of the eye. Typically, 10 to 15 percent of anterior chamber IOL implantations have to be removed for improper fitting. A low-compression IOL of the current invention is superior to a traditional IOL in that it is compatible with an increased range of anterior chamber diameters so that a one-size-fits-all IOL can be used. Such an IOL eliminates the need for a clinician to guess which size IOL to use to correct refractive errors, and also eliminates the need to carry multiple sizes of IOLs in clinical stock.

The low-compression intraocular lens described herein is useful for the surgical treatment of optical distortions that reduce visual acuity in a human eye. To understand the utility of the disclosed invention it is helpful to have a grasp of the structural features of the human eye.

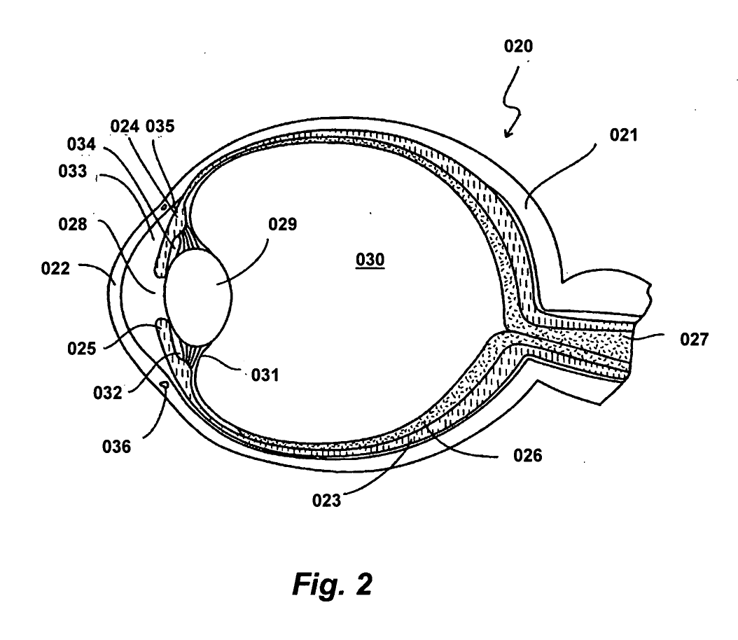

FIG. 2 illustrates a cross-section of a human eye 020 and depicts many of the features of the eye's anatomy. The eye 020 is composed of three layers: an outer layer composed of a thick sheath called the sclera 021 covering the posterior bulk of the eye, and a transparent covering called the cornea 022 over the anterior 1/6; a middle layer called the choroid 023 posteriorly, containing the vasculature and musculature of the eye, joining the ciliary body 024 and iris 025 anteriorly; and an inner layer called the retina 026, comprising a nervous membrane. The layers are pierced posteriorly by the optic nerve 027 and blood vessels of the retina. The cornea 022 comprises collagen fibers arrayed in a tightly ordered fashion such that the resulting structure is substantially transparent.

The iris 025 is an opaque diaphragm having an aperture called the pupil 028 at its center, and expands or contracts the opening of the pupil 028 by contracture and relaxation of the ciliary muscle in the ciliary body 024 to regulate the flow of light into the eye 020. Pigments of the iris provide the colored portion of the eye. The natural crystalline lens 029 is suspended between the iris 025 anteriorly and the vitreous body 030 posteriorly by ligaments known as the zonules of Zinn 031 attached to the muscles of the eye 020 in the ciliary body 024. At the junction between the iris 025 and the ciliary body 024 is a shallow depression known as the ciliary sulcus 032. An aqueous fluid filed anterior chamber 033 separates the posterior endothelial layer of the cornea from the iris. A prosthetic anterior lens can be implanted into this chamber. The iris 025 and pupil 028 divide the anterior region of the eye 020 into the anterior chamber 033 and the posterior chamber 034, which are filled with the aqueous humor, a fluid secreted by the ciliary process and flowing from the posterior chamber 034 through the pupil 022 into the anterior chamber 033. At the angle 035 of the anterior chamber 033 (at the junction of the cornea 022, and the iris 025), the fluid is filtered through the spaces of Fontana and the pectinate villi and drains through the sinus venosus sclerae, or canals of Schlemm 036. The lens 029 is contained within a thin membrane called the lens capsule (not shown).

Light passes through the cornea 022 and the iris 028, is focused by the crystalline lens 029 to form an image on the retina 026 which then transmits the detected electromagnetic radiation to the optic nerve and ultimately to the brain for processing. Six extra-ocular muscles that rotate the eye (not shown) are attached to the outside of sclera.

Errors in visual acuity results from a number of different causes, including refractive errors that can be corrected with visual prostheses (lenses). Nearsightedness (myopia), farsightedness (hyperopia), presbyopia (loss of accommodation) and astigmatism are the most common refractive errors. The low-compression, foldable intraocular lens (IOL), of which various embodiments as disclosed herein, provides a means to correct such refractive errors The IOL can be folded for insertion through a small corneal incision, with subsequent unfolding when in place in the eye. The implanted IOL allows the flow of nutrient bearing fluids (aqueous humor) for eye health. The IOL can accommodate a large range of anterior chamber diameters with reduced shifting of the axial position of the lens and consequent optical performance degradation, and without excessive haptic pressure on the eye.

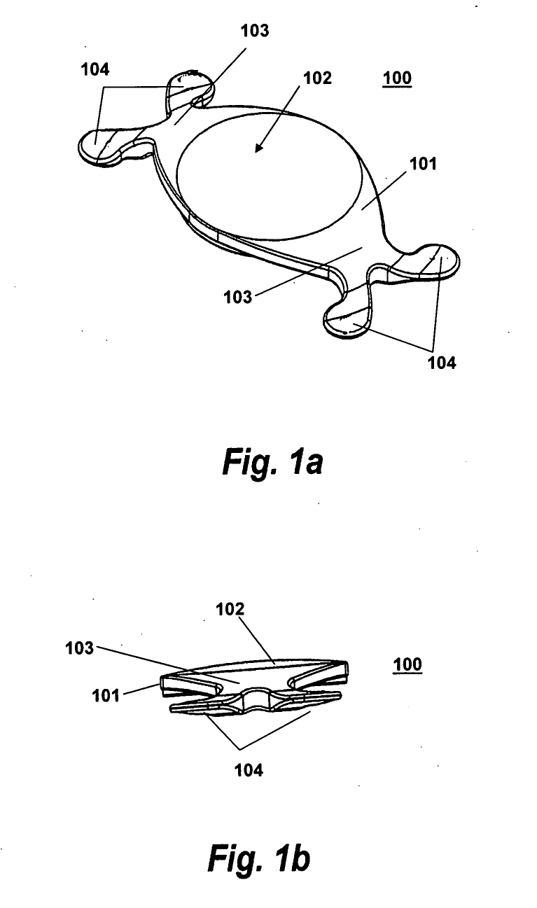

FIG. 1a illustrates a perspective top view of an IOL embodiment of the invention. In this embodiment, haptic body 100 comprises a haptic base section 101 transitioning through haptic tail regions 103 to haptic pads 104. Optical lens 102 is held by haptic base 101. In one embodiment, optical lens 102 and the haptic body 100 can be fabricated from a single piece of suitable, biocompatible optical material. In alternative embodiments optical lens 102 and the haptic body 100 may be made of different materials and subsequently assembled. Such assembled embodiments can be used to avoid tradeoffs between the mechanical properties desired for the haptic body 100, and the optical properties desired for the optical lens 102. Lens 102 may be held by haptic base section 101 through a variety of means that are well known to one of ordinary skill in the art. For example, lens 102 may be held in place by compressive force from the haptic base 101, optionally with a circumferential tongue and groove configuration. Alternatively or additionally, optical lens 102 and the haptic body 100 may be held together with the help of a biocompatible adhesive material. A third assembly option is to bond optical lens 102 to the haptic body 100 using thermal, compressive, thermocompressive, or solvent based welding techniques. FIG. 1b presents a perspective side view of the embodiment of FIG. 1a. Like elements have like numerical designations in both drawings.

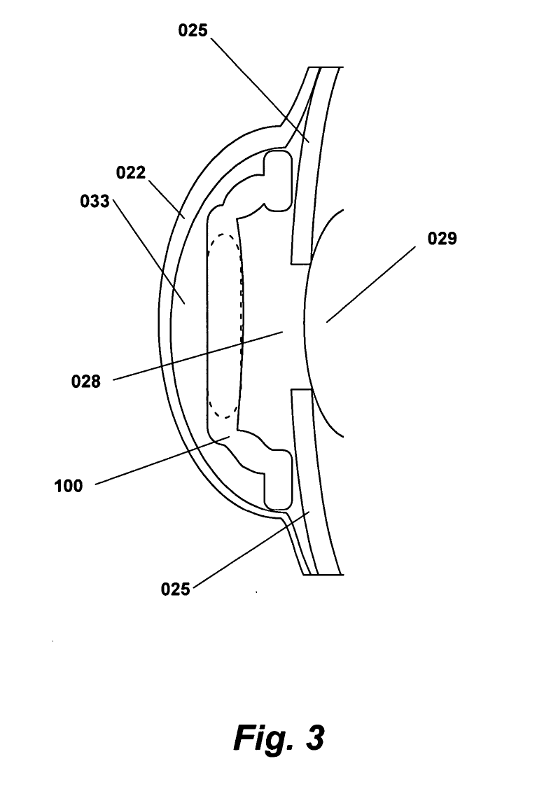

The IOL is designed for intraocular implantation in the anterior chamber 033 as shown in FIG. 3 such that it is held in position relative to the crystalline lens 029 so that light images transmitted through the cornea are corrected for proper presentation to the retina. It is desirable to fix the position of the IOL along the optical axis for maximum optical correction. Also it is desirable that IOL 100 does not interfere with adjacent eye structures 022 and 025 when implanted.

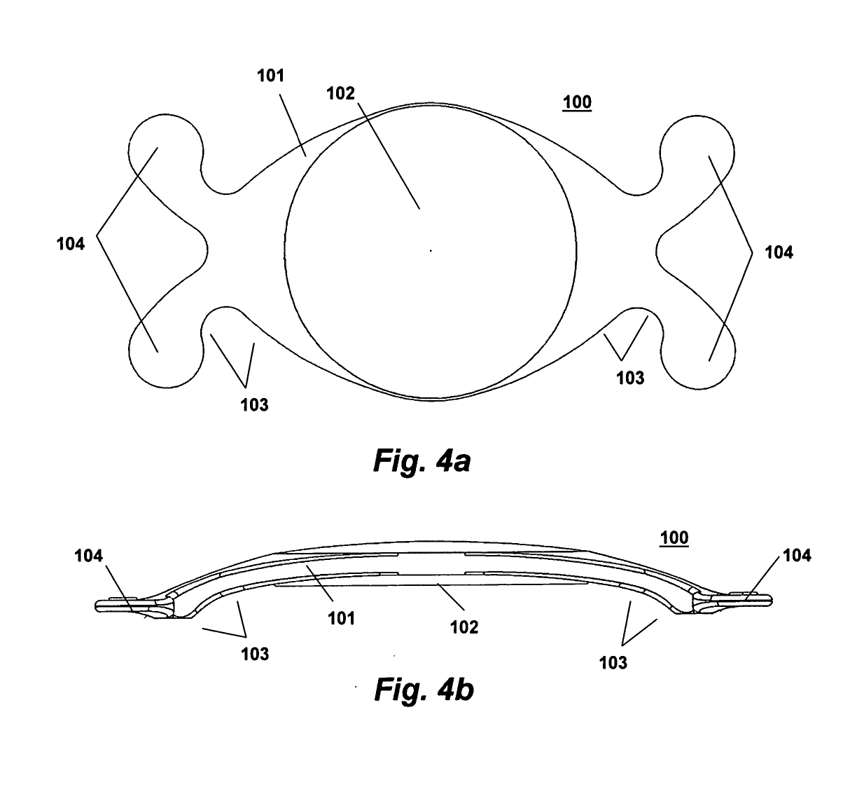

FIGS. 4a and 4b show top and side cross-sectional views, respectively of an IOL embodiment of the current invention. Numbered elements of the drawings refer to the same numbered elements as those of FIGS. 1a and 1b. Haptic tail section 103 is configured in cross section so that it can radially compress with limited vaulting of haptic base 101.

As seen in FIG. 4b, the haptic tail 103 is positioned at an angle relative to the haptic pads 104 and the lens body 101. This angle vaults or elevates that lens body 101 above the iris. For conventional IOLs, the angle of curvature of the lens, and thus the change of angle between the haptic body and the haptic pads can be 20 degrees or more, increasing with increasing radial compressive force on haptic pads 104. At such extreme angles, the vaulting can substantially displace the optical center of the optical lens along the optical axis, resulting in a degradation of optical correction. Also if extreme enough, the vaulting can cause the optical lens to contact the inner surface of the cornea, resulting in irritation and other side effects.

For the embodiment of the present invention illustrated in FIG. 4, the vault is largely independent of the compression on haptic pads 104. Thus, once positioned, the optical lens 102 is held substantially stationary relative to the crystalline lens at an appropriate position along the optical axis. The substantially stationary placement of the lens body relative to the crystalline lens provides for a more accurate correction of refractive errors as compared to a conventional intraocular lens. When inserted into the anterior chamber, the haptic pads 104 are inserted into the angle of the anterior chamber (the region in the anterior chamber where the cornea and iris join). Proper placement of the device into the anterior chamber is important because of the functional role the angle of the anterior chamber plays in eye health.

The angle of the anterior chamber contains several structures that make up the eye's drainage system. The angle is bound by the outermost part of the iris, circular fibers of the ciliaris (ciliary body), the trabecular meshwork, and the scleral venous sinus (Canal of Schlemm). Aqueous fluid flows from behind and through the iris into the anterior chamber. The aqueous fluid is drained from the anterior chamber through structures in the angle, such as the trabecular meshwork, through the scleral venous sinus. The production and drainage of aqueous fluid determines the eye's intraocular pressure (IOP). Obstruction of the angle, known as angle closure, results in elevations in IOP, which can be damaging to the health of the eye. Accordingly, proper placement of the device into the angle of the anterior chamber is important, among other reasons, to maintain proper IOP.

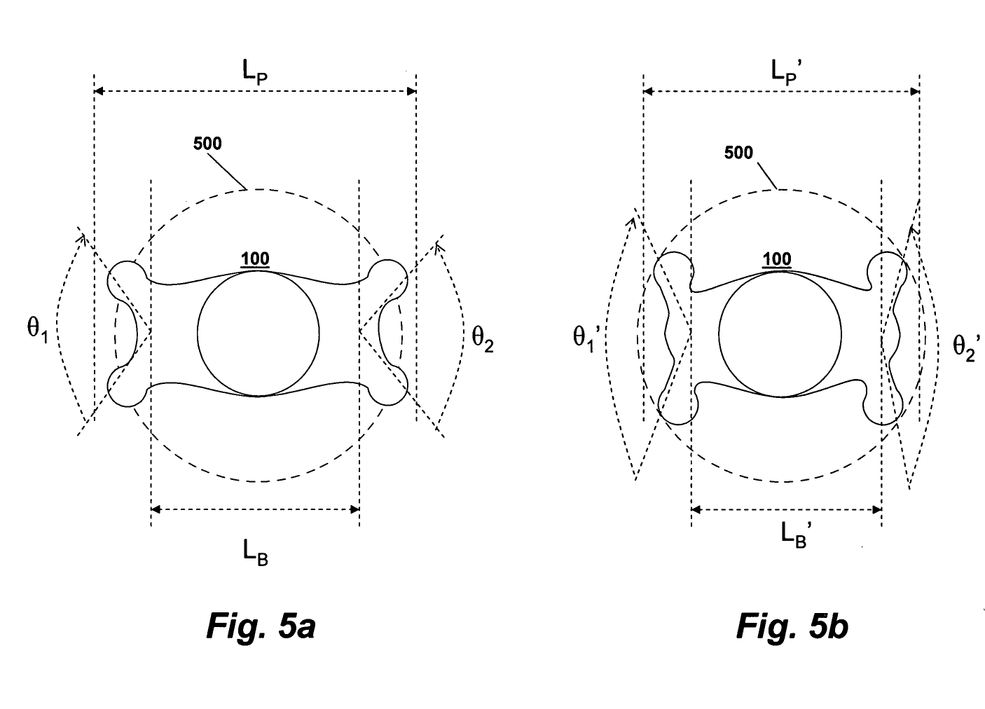

Features of the haptic tail 104 permit the installation of the device into the anterior chamber of the eye without unnecessarily blocking aqueous fluid from departing the anterior chamber. For example, in reference to the embodiment of FIG. 5b, the splay angles, θ₁′ and θ₂′, of the haptic pads are limited to retain a passage by which aqueous fluid can pass through the device and to the drainage structures of the eye. Accordingly, the design of the haptic tail prevents or at least minimizes angle closure and unacceptable IOP elevation.

FIG. 4b shows a side view of an embodiment of an IOL. From this side view, one can visualize the transition 103 from the haptic tail 104 up through the haptic base 101 to the optical lens 102. Note the angle of the haptic base leading to optical lens 102. The angle of the haptic base relative to the haptic pads 104 and the lens provides the vaulting to the IOL, holding it away from the crystalline lens. Radially compressive force from the inner wall of the anterior chamber is transmitted to the IOL through haptic pads 104, causing the pads 104 to splay apart while holding the IOL substantially stationary relative to the crystalline lens. The placement of the haptic pads 20 (FIG. 4b) relative to the haptic base 101 (FIG. 4b) provides a number of advantages to the presently disclosed devices as compared to prior art devices. For example, the haptic base 101 is offset relative to the haptic pads 104 provides a pre-determined vault to the lens body such that the lens body 102 is lifted away from the iris. Preferably, an IOL in the anterior chamber avoids contact with the corneal epithelium as well as with the iris.

Referring again to FIG. 4b, haptic tail sections 103 are configured in cross-section with thickness tapering down toward the haptic base 101. These tail sections 103 can compress under radially compressive forces, without substantially increasing the vault of haptic base 101. Thus there are two simultaneous mechanisms of compliance of the IOL to accommodate different anterior chamber diameters. The first mechanism is the splaying of the haptic pads. The second is radial compression of the haptic tail.

FIGS. 5a and 5b illustrate the two mechanisms of compliance in a schematic top view of the IOL. Dashed circle 500 indicates the interior wall of the anterior chamber that contacts the haptic pads. FIG. 5a depicts an embodiment prior to implantation, and FIG. 5b depicts the embodiment after implantation. Angles θ₁ and θ₂ are the splaying angles of the haptic pads prior to implantation, and angles θ₁′ and θ₂′ are the splaying angles after implantation. Nominally θ₁ may be approximately equal to θ₂, and θ₁′ may be approximately equal to θ₂′, but this is not a necessary condition. The splaying accommodation results from θ₁′ being greater than θ₁ and θ₂′ being greater than θ₂, as illustrated. However this splaying is not the only mechanism responsible for the decrease of L_{P} to L_{P}′ to accommodate the diameter of anterior chamber 500 as shown in FIGS. 5a and 5b, respectively. The second mechanism is the radial compression of the haptic tails as illustrated by the reduction of L_{B} to L_{B}′ in FIGS. 5a and 5b, respectively.

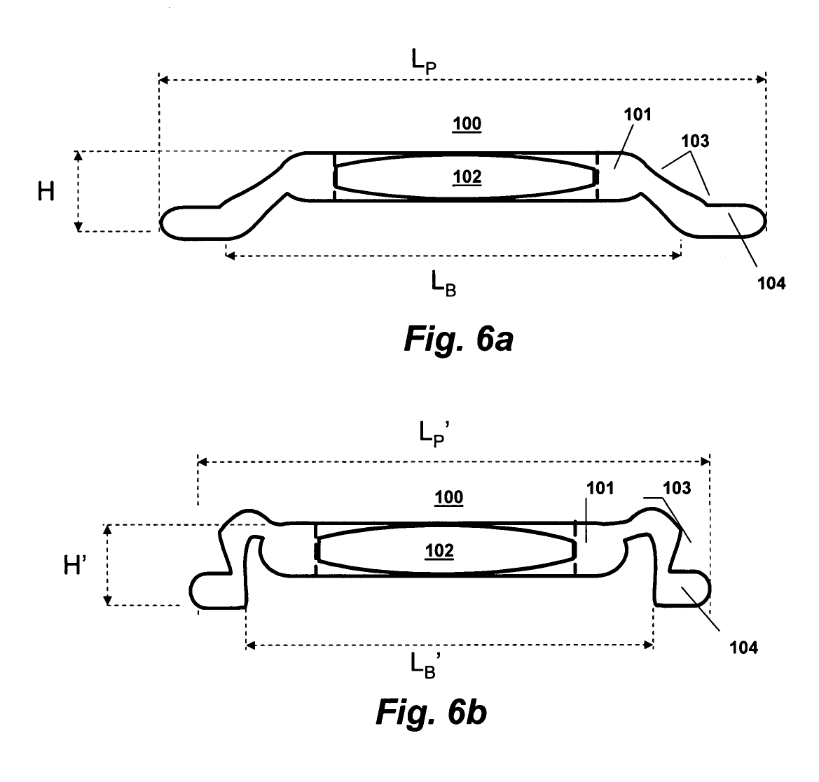

The radial compression is better understood in the cross-sectional side view schematic drawings of FIGS. 6a and 6b. Haptic tails 103 distort into more pronounced “S” or “Z” shapes with radially compressive force (i.e. haptic pads 104 being forced toward one another). The haptic tail distortion accommodates the reduction of L_{B} to L_{B}′ with a minimum change of optical lens 102 position (i.e. H is approximately equal to H′).

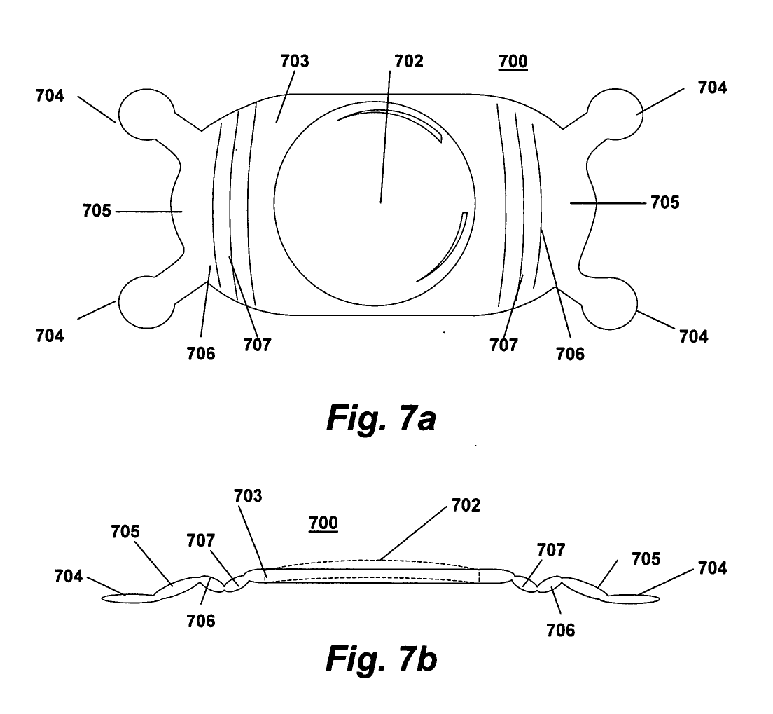

FIGS. 7a and 7b illustrate top and cross-sectional side views, respectively, of another embodiment of the invention. Numbered elements correspond in both drawings. 703 is the haptic base, that holds optical lens 702. Haptic pads 704 are as described above with respect to the embodiments of FIGS. 1, 4, 5, and 6. However, in this embodiment, haptic tails 705 are not connected directly to haptic base 703, but instead are connected via distal and proximal strain relief segments 706 and 707, respectively. The haptic pad accommodation mechanism, as described above, can apply to this embodiment. The haptic tail compression mechanism, as described, can also apply to this embodiment. This embodiment has a further accommodation mechanism in the form of the strain relief segments.

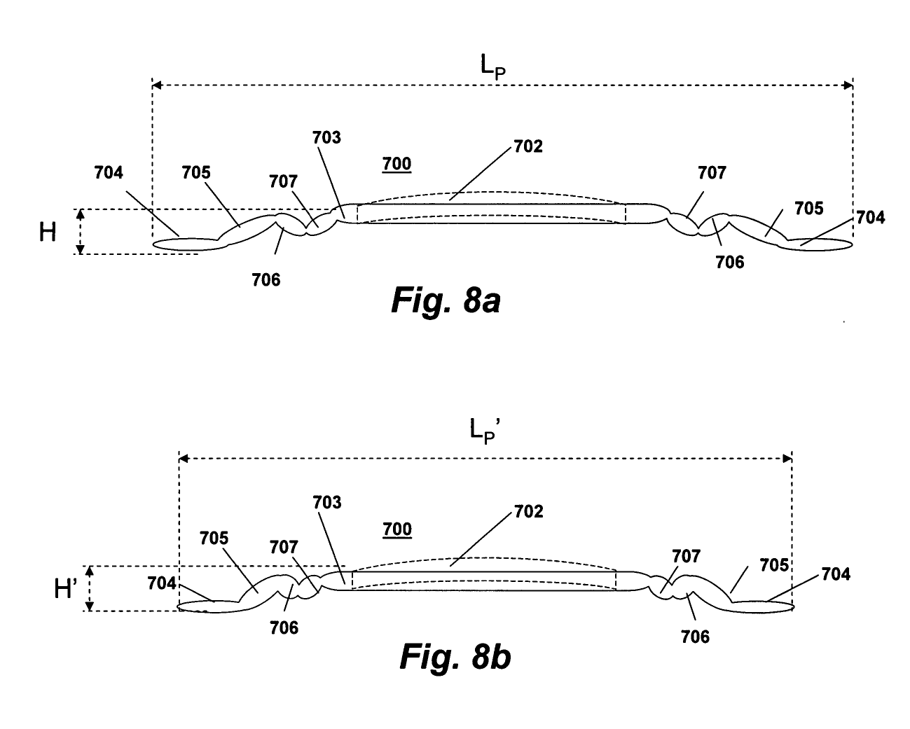

The operation of distal and proximal strain relief segments 706 and 707 is better understood in connection with relaxed and compressed cross-sectional side view drawings, FIGS. 8a and 8b, respectively. Note that in response to compression, the effective joint between distal segment 706 and proximal segment 707 is forced downward to counter the tendency of the effective joints between haptic tail 705 and distal segment 706, and proximal segment 707 and haptic base 703 to move upward. This stabilizes the vertical position of haptic base 703 (i.e. H is approximately equal to H′) as the IOL is radially compressed allowing for an even greater range of L_{P} (or correspondingly a greater range of anterior chamber diameters). In other embodiments, additional strain relief segments may be introduced to further increase the L_{P} range accommodation.

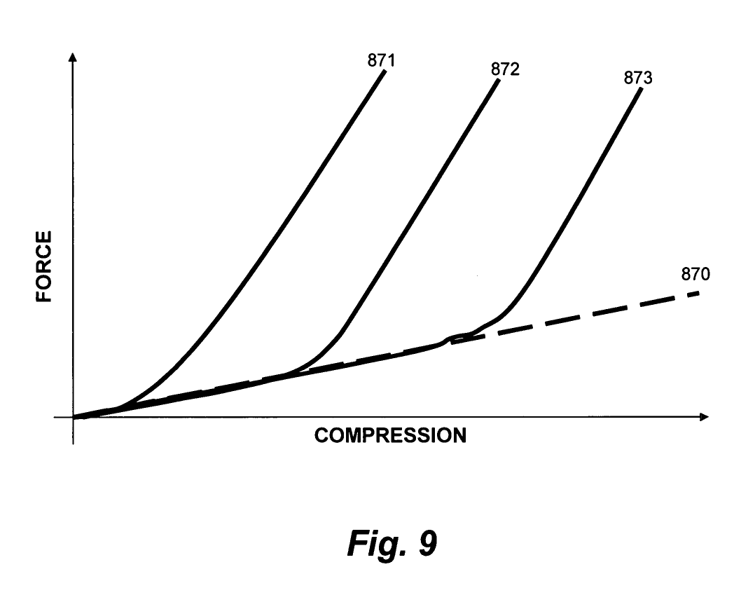

FIG. 9 is an exemplary plot of force versus radial compression characteristics for IOLs. Straight line 870 is a reference characteristic, according to Hook's law for an ideal spring, F=K C, which simply states that force and compression (measured as displacement) are proportional. In real elastic structures such as IOL haptics, the force-compression characteristics invariably deviate from ideality because of structurally related mechanical constraints. With IOL haptics, for example, the physical size of elastically deforming structures tends to limit the mechanical range of ideal-like force-compression behavior. By including additional mechanical accommodation mechanisms, as described above, the range of ideal-like force-compression behavior can be extended.

For example, In FIG. 9 curve 871 represents an IOL force-compression characteristic for an IOL embodiment with only a pad splaying accommodation mechanism. Haptic pads can only splay so much, after which it becomes harder and harder to splay them further as shown for curve. Curve 872 represents a force-compression characteristic for an IOL embodiment with pad splaying and haptic tail compression accommodation mechanisms. By combining the two mechanisms, the range of more ideal force-compression behavior is extended. This embodiment can accommodate a larger range of anterior chamber diameters, without exerting excessive force (pressure) on eye structures. Curve 873 represents a force-compression characteristic for an IOL embodiment with pad splaying, haptic tail compression, and strain relief segment accommodation mechanisms. The addition of the strain relief segment accommodation mechanism further extends the compression range for ideal-like force compression behavior, further extending the range of anterior chamber diameters that can be accommodated.

The above embodiments show how using multiple elastic structural elements and/or multiple mechanical modalities for deforming those elements can extend the range of ideal-like force-compression behavior, without increasing the physical size of the IOL. For example, the pad splaying occurs tangentially in a plane normal to the optical axis, whereas the tail compression occurs radially in a plane normal to the optical axis.

Although all of the IOL embodiments described so far have two diametrically opposed haptic tails, in general, an arrangement of any number of haptic tails may be used. For example, embodiments with three, four, or more tails can be considered. In particular, an embodiment with three or more tails can provide improved IOL fixation in a plane normal to the optical axis. A drawback of using more than two haptic tails is a potential reduction in the flow of aqueous humor. Also, such embodiments may be more difficult to fold during implantation. The present invention provides the flexibility to make such tradeoffs for different clinical cases.

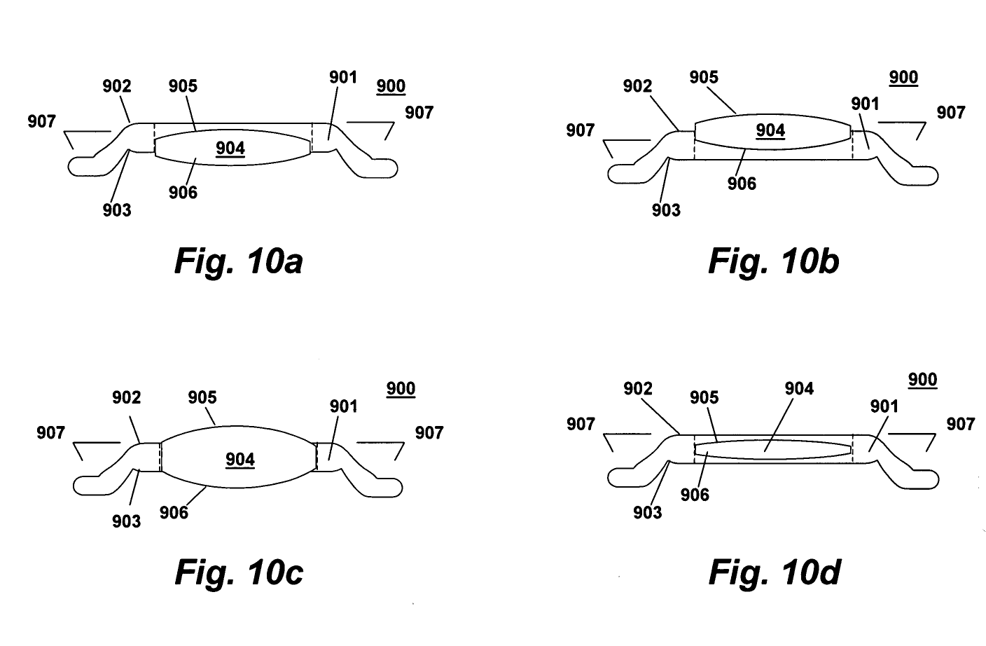

FIG. 10a shows an exemplary assembly of an optical lens 904 on a haptic base 901, having a front side 902 and a back side 903, of a haptic body 900. The optical lens 904 has a front side 905 and a back side 906. The front side 905 faces the cornea and the back side 906 faces the pupil. In the embodiment shown in FIG. 10a, the optical lens is offset to the back side of haptic body. In another embodiment (FIG. 10b), the optical lens can offset to the front side of haptic body. Alternatively, in further embodiments, the optical lens may extend beyond both sides of the haptic base (FIG. 10c) or be recessed from both sides of the haptic base (FIG. 10d).

A particularly preferred embodiment of the disclosed device is the embodiment of FIG. 9a in which the optical lens 904 is offset into the space between the back side of the haptic body 900 and the pupil (not shown). This preferred placement of the optical lens allows additional clearance space between the corneal epithelium and the front side 905 of the optical lens. Clearance between the posterior portion of the optical lens 904 and the iris is provided by the angle of the haptic tails.

The corrective devices disclosed herein use any type of light focusing technology in the lens body. Thus, disclosed corrective devices can use refractive optics, diffractive optics, combinations of refractive and diffractive optics, and other types of optics systems as embodiments of the lens body. Use of refractive lens technology is exemplified in U.S. Pat. No. 5,089,022, to Koester, et al., entitled “Rectified intraocular lens,” which issued on Feb. 18, 1992 and is incorporated by reference in its entirety.

Diffractive lens technology is another viable optical system for use with the disclosed invention. One example of diffractive optics that can be used with the present invention is found in U.S. Pat. No. 5,152,787, to David Hamblen, entitled “Intraocular gradient-index lenses used in eye implantation,” which is hereby incorporated by reference in its entirety. Additional examples of diffractive lens technology used in intraocular lenses include U.S. Pat. Nos. 5,120,120, 5,358,520, 5,366,502, 5,384,606, 5,448,312, 5,485,228 and 6,634,751, all of which are hereby incorporated by reference in their entirety.

A preferred diffractive lens technology is disclosed in U.S. Pat. No. 5,589,982 to Faklis, et al., entitled “Polychromatic diffractive lens,” which is hereby incorporated by reference. This multi-order diffractive (MOD) lens technology can be used to construct the optical portion of the lens body. An MOD lens focuses light of different wavelengths to a multiple order diffractive singlet. The multi-order diffractive structure has a plurality of annular zones, which define zone boundaries that diffract light of each of the wavelengths in a different diffractive order to the focus thereby providing a plural or multiple order diffractive singlet. MOD lenses are superior to other lens technologies in that they focus light with less image distortion.

The disclosed corrective devices can be used with a monofocal, bifocal or polyfocal corrective optic. In one embodiment, a monofocal MOD corrective optic is used and focuses light comprised of multiple wavelengths to a single focal point. Accordingly, a bifocal MOD corrective optic focuses light comprised of multiple wavelengths to two focal points, while a polyfocal MOD corrective optic focuses light to three or more focal points. For example, recent advances by Morris, et al., U.S. patent application Ser. No. 10/462,294, entitled, “Bifocal Multiorder Diffractive Lenses for Vision Correction,” which is hereby incorporated by reference in its entirety, describes the construction of bifocal and polyfocal MOD lenses. It is noted that other types of lens technology can be used to produce monofocal, bifocal and polyfocal lenses.

It should also be noted that by varying the central optical zone of the MOD lens, different powers of the resulting lens body 130 can be manufactured. A higher MOD number permits a larger central “optical zone”. MOD optical zones will preferable range from 1 to 20 unites. More preferably from 10 to 20, and most preferably the lens body will have a MOD number of 10. By varying the nature of the MOD lens a progress set of lenses are produced that achieve the optimum contrast sensitivity for patients with varied pre-operative refractive errors.

The thickness of the optical lens 904 is an important feature of the described devices but its importance is reduced because of the haptics described herein. In general, it is preferred to minimize the overall size of an IOL to reduce unintended alterations to the shape of the cornea during the installation procedure. However, so long as the optical lens 904 itself avoids contact with the corneal epithelium and the iris, the optical lens 904 can be as thick as is necessary to restore visual acuity to an eye. Exemplary thicknesses of an optical lens range from approximately 25 to 1000 microns, 50 to 600 microns, 75 to 250 microns, and most preferably, the optical lens will be approximately 100 microns thick.

The optical lens 904 preferably has a diameter of at least 3 millimeters. A preferred lens body has a diameter from approximately 3 to 10 millimeters. Specific examples of diameters for the lens body include diameters of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 millimeters.

Another feature of the optical lens 104 relates to its shape. The optical lens 904 can be flat or curved. The optical lens 904 can be positions above the haptic structure 900 (FIG. 904b), below the haptic structure 900 (FIG. 904a), or flush with the top side, bottom side or both sides of the haptic structure. When the optical lens 904 is curved, the curvature may be concave or convex. The optical lens 904 may also be plano-concave or plano-convex. It is preferred that the optical lens 904 has clearance below the corneal epithelium and above the iris.

The optical lens 904 assembled with haptic structure 900 preferably has a radius of curvature that provides a degree of flexibility for the IOL during and after installation. The radius of curvature will preferably range from approximately 1 to 50 mm, 5 to 40, 10 to 30, and most preferably, the radius of curvature of the optical lens 904 is approximately 12.5 mm. The radius of curvature for an optical lens is preferably established ex vivo and the ranges discussed above relate to radii of curvature from lenses that are not installed within an eye.

The dimensions of the described corrective devices will also depend on the nature of haptic structure 900. One IOL embodiment is constructed of materials that produce a device will preferably be 7 to 20 millimeters in length and from 3 to 10 millimeters in width.

Unlike WO 02/41806, which teaches the use of MOD lens technology for the construction of a thin, foldable intraocular implant, the presently described invention does not require such thin lenses. With respect to intraocular lenses, the present invention contemplates intraocular lenses wherein the lens structure can be of any thickness, so long as the resulting structure fits efficaciously and safely within the anterior chamber of an eye. In one embodiment, the thickness of the lens is greater than 125 microns. More specifically, the thickness of the monofocal lens embodiment will be 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450 or more microns in thickness. These thickness ranges preferably apply to embodiments comprising a monofocal MOD corrective lens.

While particular optical lens thicknesses are not required for optical performance, some embodiments may benefit from possessing a optical lens with a certain degree of thickness. Such a construction may serve to help stabilize the IOL, thus better holding the optical lens in a stable position relative to the retina of the eye in which the lens is installed. Additionally, an increased thickness of an IOL may prevent lens deformation once the lens is installed in a subject's eye. Accordingly, increased lens thickness may provide less distortion in the transmission of light through an intraocular lens to the retina.

The disclosed optical lenses are composed of a optically transmissive material, such as typically used in the manufacture of contacts, optic portion of conventional IOLs, spectacles, or other types corrective lenses (e.g., plastic, silicone, acrylic, glass, or polymers typically used for the particular contact, IOL, or spectacle application). Such lenses can be constructed using typical methods that are well known to one of ordinary skill in the art, for example: grinding, lathing, etching, molding, or combinations thereof. For example, the optic pin of a corrective device can be prepared by lathing.

When an IOL is designed for anterior chamber installation, it preferably comprises a lens body 904 and a haptic body 900, upon which the lens body is affixed, attached, or otherwise positioned. A unitary design where the lens body 904 and the haptic body 900 comprise a single continuous unit is also contemplated.

The haptic body 900 is preferably made of flexible silicone such as Material Number MED-6820 commercially available from NuSil Silicone Technology of Carpenteria, Calif. Other resilient materials such as PMMA or hydrogel can be used. The haptic body 900 serves as a scaffold to support and position the optical lens 904 and preferably has no corrective properties. In alternative embodiments, the haptic body 900 can be formed to enhance or contribute to the corrective effect of the optical 904.

Additionally, haptic body 900 can be coated with materials that render it less likely to interact with components of the eye. An example of such a coating would be a coating of heparin, an natural saccharide that inhibits blood clotting and protein adhesion. The haptic body material also comprises one or more ultraviolet light blocking agents.

The haptic body 900 is preferably made in a thickness of approximately 25-1000 microns, alternatively the membrane is from 50 to 600 microns, 75 to 250 microns, and most preferably, the optical lens 904 will be approximately 100 microns thick. Although flexible, it returns to a rest position with a radius curvature in a range of approximately 0 to 20 millimeters about a vertical axis. The range includes a radius curvature of 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0 millimeters about a vertical axis. The combined layers exhibit a total thickness of about 475 plus or minus 10 microns. The overall dimensions are approximately 12 millimeters in length, 8 millimeters in width. The IOL can be bent and even rolled or folded for insertion into the interior chamber through a small incision of preferably no more than 3.0 millimeters, more preferably 2.75 millimeters in length or less. The IOL has enough resiliency to return to its prerolled or prefolded arcuate shape.

The IOL retains an arcuate shape in its resting position. The IOL within a circle having a radius R of approximately 12.5 millimeters. Due to the range of accommodation for haptic body 900, this size can accommodate practically all eye sizes. In other words, depending upon the span of the anterior chamber, the IOL upon installation can adjust its length by decreasing or increasing its radius R of curvature within a range of about 0 to 20 millimeters.

The IOL can be used in an aphakic, pseudophakic or phakic eye to correct impaired vision. For example, the disclosed lenses can be used in lens replacement procedures, such as for use in an aphakic eye. So, after the procedure the procedure the subject's eye would contain a plurality of lenses, including but not limited to the natural lens and at least one corrective lens or a plurality of IOLs. In a preferred embodiment, a multiorder MOD corrective lens is designed to correct a refractive error. Typically, the MOD corrective lens is fashioned after the nature and degree of the refractive error in a subject has been determined. The corrective lens can be monofocal, bifocal, or polyfocal. The MOD corrective lens, once fashioned, is provided to the subject, alleviating the refractive errors present in the uncorrected eye.

As discussed above, the dimensions for the corrective device are governed by the intended use of the device. For example, one IOL embodiment is designed for installation and use within the anterior chamber of the eye.

The size of the folded IOL is important because it is well known in the art that incisions for installing IOLs that are larger than 5 millimeters in length tend to induce astigmatism or other distortions of the cornea that may themselves lead to visual impairment. Accordingly, it is preferable that an intraocular corrective device intended for insertion into the anterior chamber of an eye will be adequately flexible. Preferably, such a device will be sufficiently flexible as to be foldable for insertion into an incision of preferably less than or equal to 4.0 millimeters in length. An incision size of 1.0 to 5.0 millimeters or less is most preferred. Specifically, an incision size of 1.0, 1.1, 1.2, 1.25, 1.3, 1.4, 1.5, 1.6, 1.7, 1.75, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0 millimeters in length. Sutures or other wound closing agents, such as glues, adhesives, protein cross-linking agents, and the like may or may not be used to close the incision.

The disclosed embodiments are implanted in the anterior chamber of the eye. The disclosed embodiments can be used alone or in combination with another IOL to correct vision. For example, a bifocal IOL of the disclosed design can be used in the anterior chamber to provide additional correction in an eye where the crystalline lens had been removed and replaced with a posterior chamber IOL.

The following examples are offered to illustrate but not to limit the invention.

### EXAMPLE 1

### Phakic Intraocular Lens (IOL) Implantation and Explantation

A foldable phakic IOL is implanted using an injector that introduces the IOL through a clear corneal incision of less than 3 mm. The implantation technique for this lens is similar to that used for a pseudophakic IOL after cataract extraction. Preopertively, topically instilled pilocarpine 1% is administered to create a miotic pupil. The surgeon leads the phakic IOL into the lubricated injector cartridge, creates a sideport incision, and injects a viscoelastic agent into the anterior chamber. The IOL is then injected. The surgeon engages the inferior haptics of the IOL into the inferior angle before removing the cartridge tip from the anterior chamber. Bimanual irrigation/aspiration (I/A) removes all viscoelastic from the anterior chamber, and the surgeon uses the I/A instruments to adjust the position of the lens, if necessary. The anterior chamber is inflated to a normal pressure with BSS (Alcon Laboratories, Inc. Ft. Worth, Tex.), and the incision is checked. Finally, the surgeon places a bandage contact lens and a drop of ZYMAR (Allergan, Inc., Irvine, Calif.) on the eye. The entire surgical procedure takes only a few minutes, and can be performed using only topical anesthesia in an outpatient setting. Following implantation, the subject immediately enjoys improved visual acuity.

The surgeon may explant the IOL by grasping its superior haptic with forceps through an incision and then externalizing the entire IOL by means of gentle traction. Sutures or other methods for sealing the incision may be used.

1. An intraocular lens comprising:
a lens body;
a haptic base, comprising a proximal end and a distal end, wherein the proximal end of the haptic base is connected to the lens body by a first vertically flexible junction region, wherein the first junction region flexes vertically relative to the lens body; and
a haptic tail, wherein the haptic tail comprises a haptic footplate connected to the haptic tail by a horizontally flexible member, wherein the horizontally flexible member flexes horizontally relative to the lens body, and wherein the distal end of the haptic base is connected to the haptic tail by a second vertically flexible junction region, wherein the second junction region flexes vertically relative to the lens body.

2. The intraocular lens of claim 1, wherein the lens body, the first junction region, the haptic base, the second junction region and the haptic tail form an S-curve.

3. The intraocular lens of claim 1, wherein the lens body and the haptic base form meet to form an acute angle at the first junction region and the haptic base and the haptic tail meet to form an acute angle at the second junction region.

4. The intraocular lens of claim 3, wherein the angle at the first junction region and the angle at the second junction region decrease upon implantation of the intraocular lens in an eye of a recipient.

5. The intraocular lens of claim 4, wherein the angle at the first junction region and the angle at the second junction region increase after the intraocular lens adapts to the eye of a recipient.

6. The intraocular lens of claim 5, wherein the angle at the first junction region and the angle at the second junction regions stabilize from 1 to 6 hours after implantation.

7. The intraocular lens of claim 1, wherein upon implantation of the intraocular lens in an eye of a recipient, the haptic footplate moves horizontally while the first and second vertically flexible junction regions move vertically, thereby minimizing vertical vault of the lens body.

8. The intraocular lens of claim 1, wherein the haptic footplate comprises a width that is greater than that of the flexible member.

9. The intraocular lens of claim 1, wherein the lens body further comprises an optic.

10. The lens of claim 9, wherein the optic is a refractive optic.

11. The lens of claim 9, wherein the optic is a diffractive optic.

12. The lens of claim 11, wherein the diffractive optic is a multi-order diffractive structure having a plurality of zones which define zone boundaries at which light incident on the structure experiences an optical phase shift, and diffracts light of each of the wavelengths in a different diffractive order, m, such that m≧1, to said focus, thereby providing a plural order diffractive singlet.

13. The lens of claim 9, wherein the optic comprises a refractive component and a diffractive component.

14. An intraocular lens comprising:
a lens body;
a haptic base, comprising a proximal end and a distal end, wherein the proximal end of the haptic base is connected to the lens body by a first vertically flexible junction region, forming a first angle; and
a haptic tail, wherein the haptic tail comprises a haptic footplate connected to the haptic tail by a horizontally flexible member, wherein the distal end of the haptic base is connected to the haptic tail by a second vertically flexible junction region, forming a second angle, wherein the first angle and the second angle decrease in size upon implantation of the intraocular lens in an eye of a recipient, thereby minimizing or eliminating vertical vaulting of the lens body.

15. An intraocular lens haptic, comprising:
a junction region, having a proximal end and a distal end;
a haptic base flexibly connected to the proximal end of the junction region; and
a haptic footplate connected to the distal end of the junction region by a horizontally flexible member, wherein the junction region is wider than either the haptic footplate or the haptic base.

16. A intraocular lens, comprising:
a haptic;
a lens body in a first plane, wherein the lens body comprises an anterior surface and a posterior surface; and
an optic in a second plane, wherein the optic comprises an anterior surface and a posterior surface, wherein the first plane is different from the second plane and wherein either the anterior surface of the lens body or the anterior surface of the optic is relatively closer to the iris, but not both.

17. An intraocular corrective lens comprising:
an optic disposed within a lens body, wherein the lens body is substantially planar; and
a first haptic base extending from the lens body to a first haptic tail, wherein the lens body, the first haptic base and the first haptic tail form a first S-curve having a first region of inflection;
a second haptic base extending from the lens body to a second haptic tail, wherein the lens body, the second haptic base and the second haptic tail form a second S-curve having a second region of inflection; and
a first haptic footplate extending from the first haptic tail in a plane substantially parallel to the lens body and a second haptic footplate extending from the second haptic tail in a plane substantially parallel to the lens body,
wherein a distance between the first and second regions of inflection decrease upon implantation of the intraocular corrective lens in a recipient's eye.

18. An intraocular corrective lens comprising:
an optic with a diameter disposed within a lens body, wherein the lens body has a width approximately equal to the diameter of the optic;
at least one haptic base extending from the lens body at an acute angle comprising a proximal end and a distal end, wherein the proximal end of the haptic base has a width approximately equal to the width of the lens body; and
a haptic tail comprising at least two haptic footplates extending from the haptic base at an acute angle, wherein the footplates extend at angles less than 65° degrees out from the haptic base and wherein the footplates extend in a plane substantially parallel relative to the lens body.

19. The lens of claim 18, wherein the diameter of the optic is less than 7 mm.

20. The lens of claim 19, wherein the width of the distal end of the haptic base is less than half the diameter of the optic.

21. The lens of claim 18, wherein the at least one haptic base extends from the lens body at less than 45° degrees.

22. The lens of claim 21, wherein the at least one haptic base extends from the lens body at less than 30° degrees.

23. The lens of claim 18, wherein the optic is a refractive optic.

24. The lens of claim 18, wherein the optic is a diffractive optic.

25. The lens of claim 24, wherein the diffractive optic is a multi-order diffractive structure having a plurality of zones which define zone boundaries at which light incident on the structure experiences an optical phase shift, and diffracts light of each of the wavelengths in a different diffractive order, m, such that m≧1, to said focus, thereby providing a plural order diffractive singlet.

26. The lens of claim 18, wherein the optic comprises a refractive component and a diffractive component.

27. A haptic tail comprising:
a haptic base; and
a haptic footplate comprising an end proximal to the haptic base and a distal end, wherein the footplate extends from the haptic base at an angle less than 65° degrees, and wherein the proximal end of the footplate has a width that is approximately half that of the distal end of the footplate.

28. The haptic tail of claim 27, wherein the haptic base extends in an ascending curve from the haptic footplate.

29. The haptic tail of claim 28, wherein the haptic base extends from the haptic tail at less than 45° degrees.

30. The haptic tail of claim 29, wherein the haptic base extends from the haptic tail at less than 30° degrees.

31. An intraocular lens (IOL) comprising:
an optic defining a vertical optical axis;
a haptic base having a proximal portion in connection with the optic and extending outwardly from the optical axis through a transition portion into a distal end portion, wherein the transition portion extends vertically downward from the proximal portion through two generally opposing angles of curvature into the distal portion and acts to elevate the proximal portion from the distal portion; and
a haptic tail having a proximal portion in connection with the distal portion of the haptic body and extending generally horizontally outward into a pair of haptic footplates.

32. The intraocular lens of claim 31, wherein the haptic base comprises a thin membrane.

33. A method of implanting an anterior chamber intraocular lens, comprising:
providing an intraocular lens comprising an optic disposed within a lens body and a haptic base extending from the lens body in a descending arc with a slope to a haptic tail, wherein the haptic tail comprises a haptic footplate extending from the haptic base, wherein the slope of the arc of the haptic base increases upon implantation of the intraocular corrective lens in a recipient's eye;
creating an incision in an eye, wherein the eye comprises an anterior chamber and a chamber angle, wherein the incision is approximately less than 2 mm in length and provides access to the eye's anterior chamber; and
introducing the intraocular lens into the anterior chamber of the eye, whereby the haptic footplate is introduced into the chamber angle of the eye.

34. The method of claim 33, wherein no peripheral iridectomy is performed.
